(19) United States
(12) Patent Application Publication
(10) Pub. No.: US 2025/0179351 A1
(43) Pub. Date: Jun. 5, 2025

(54) ORGANIC LIGHT-EMITTING MATERIAL CONTAINING TETRAPHENYLBENZENE, PREPARATION,AND APPLICATION

(71) Applicant: South China University of Technology, Guangzhou (CN)

(72) Inventors: Benzhong Tang, Guangzhou (CN); Anjun Qin, Guangzhou (CN); Pengbo Han, Guangzhou (CN); Dongge Ma, Guangzhou (CN); Zeng XU, Guangzhou (CN); Zujin Zhao, Guangzhou (CN); Rongrong Hu, Guangzhou (CN); Zhiming Wang, Guangzhou (CN)

(21) Appl. No.: 17/611,418

(22) PCT Filed: Jan. 3, 2020
(86) PCT No.: PCT/CN2020/070205

§ 371 Date: Jul. 17, 2024

(30) Foreign Application Priority Data
May 14, 2019 (CN) 201910396951.8

(51) Int. Cl. C09K 11/06; H10K 50/11; H10K 85/60

(52) U.S. Cl. CPC C09K 11/06; H10K 85/631; H10K 85/654; H10K 85/6572; C09K 2211/1007; C09K 2211/1014; C09K 2211/1018; H10K 50/11

(57) ABSTRACT

The present disclosure belongs to the field of organic optoelectronic materials, and disclosed are a tetraphenylbenzene-containing organic light-emitting material, and a preparation method and an application thereof. The tetraphenylbenzene-containing organic light-emitting material has a structural formula as Formula I, where R₁ is an electron-donating group of an aromatic ringderivative, and R₂ is an electron-withdrawing group of the aromatic ringderivative. The preparation thereof includes the following steps: using 1,4-dibromo-2,5-terphenyl and R₂-substituted phenylboronic acid as raw materials, obtaining an R₂-containing aromatic ringcompound through a Suzuki reaction; and then reacting the R₂-containing aromatic ringcompound with R₁-substituted phenylboronic acid or boronic acid ester in the presence of a tetrakis(triphenylphosphine)palladium catalyst to obtain the tetraphenylbenzene-containing organic light-emitting material. The tetraphenylbenzene-containing organic light-emitting material of the present disclosure has the features of efficient sold-state luminescence and bipolarity, and can be used to prepare non-doped blue organic light-emitting devices with high efficiency and low roll-off.

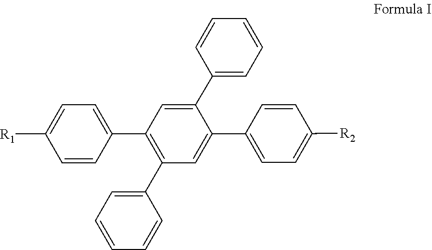

### FIELD OF THE INVENTION

The present disclosure belongs to the field of organic optoelectronic materials, and specifically relates to a tetraphenylbenzene-containing organic light-emitting material, and a preparation method and an application thereof.

### BACKGROUND OF THE INVENTION

With the rise and vigorous development of the organic electronic industry, organic optoelectronic materials have been widely used in the field of organic light-emitting devices (OLEDs). Thanks to their important scientific research value and broad commercial application prospects, the organic optoelectronic materials have become a rapidly growing field in material science. To explore and develop organic light-emitting materials with excellent performance, high luminous efficiency, and adjustable emission wavelength is an urgent problem that needs to be solved by researchers. However, it is quite difficult to develop efficient light-emitting materials because light of conventional materials will be quenched in the aggregate state.

### SUMMARY OF THE INVENTION

In view of the defects and deficiencies in the prior art, a primary objective of the present disclosure is to provide tetraphenylbenzene-containing organic light-emitting material. Tetraphenylbenzene has a simple molecular structure, and is convenient for chemical modification and functionalization. The present disclosure uses tetraphenylbenzene to form a material with high solid-state luminous efficiency. A light-emitting material with high solid-state luminous efficiency and bipolar transmission characteristics can be obtained by combining tetraphenylbenzene with a hole transport material and an electron transport material. These materials have shown excellent performance in the field of organic electroluminescence. In general, tetraphenylbenzene is a functional group with simple structure, excellent performance, and aggregation-induced emission characteristics, and has broad application prospects in the formation of organic optoelectronic materials.

Another objective of the present invention is to provide a preparation method of the above tetraphenylbenzene-containing organic light-emitting material.

Still another objective of the present invention is to provide an application of the above tetraphenylbenzene-containing organic light-emitting material in organic light-emitting devices.

The objectives of the present disclosure are achieved by the following technical solutions.

A tetraphenylbenzene-containing organic light-emitting material has a structural formula as Formula I:

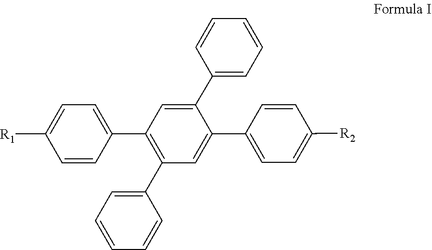

where, R₁ is an electron donating group of an aromatic ringderivative, and R₂ is an electron withdrawing group of the aromatic ringderivative.

Further, the R₁ is one of the following substitutional groups from 1 to 20:

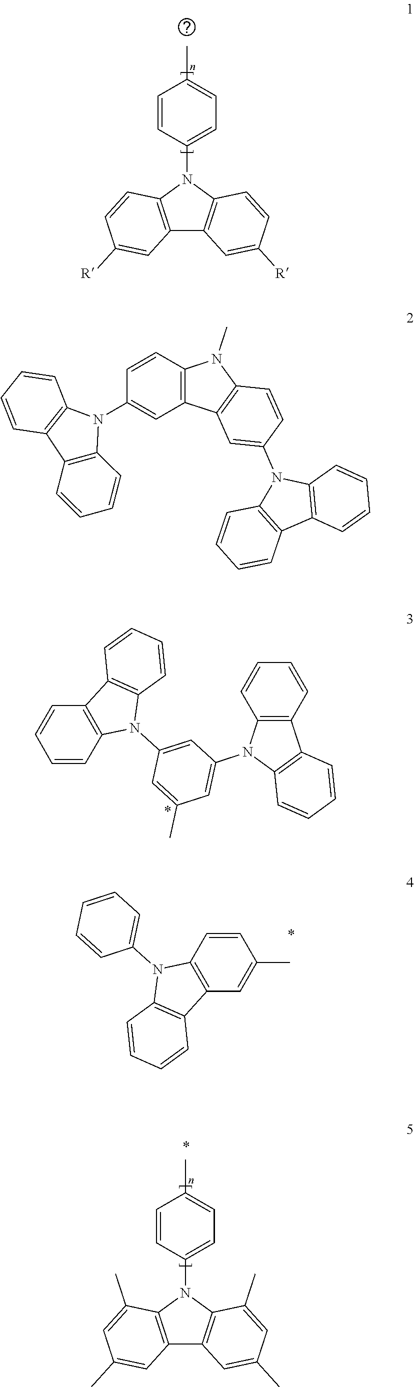

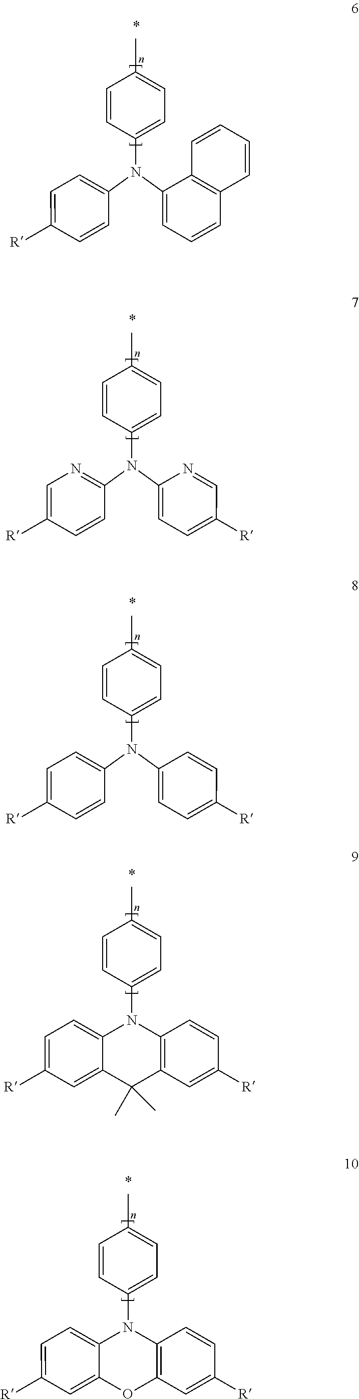

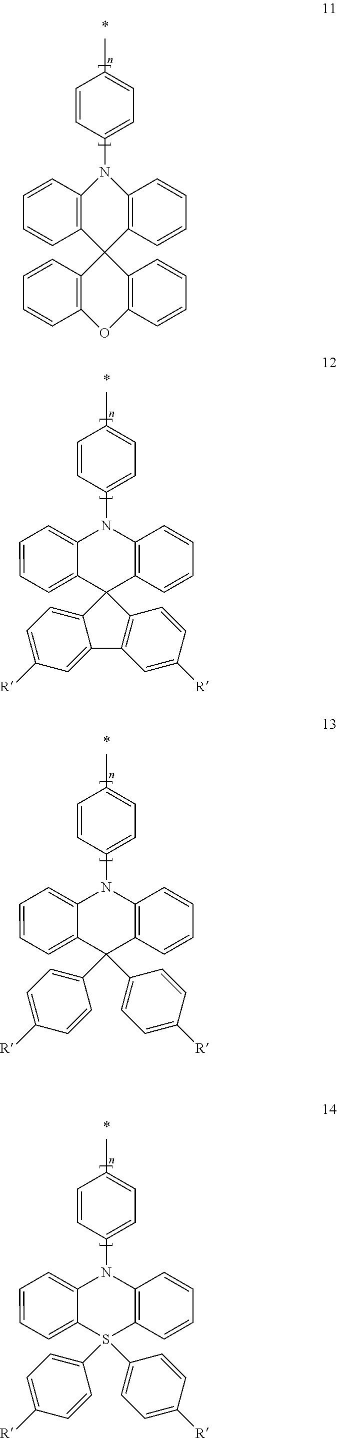

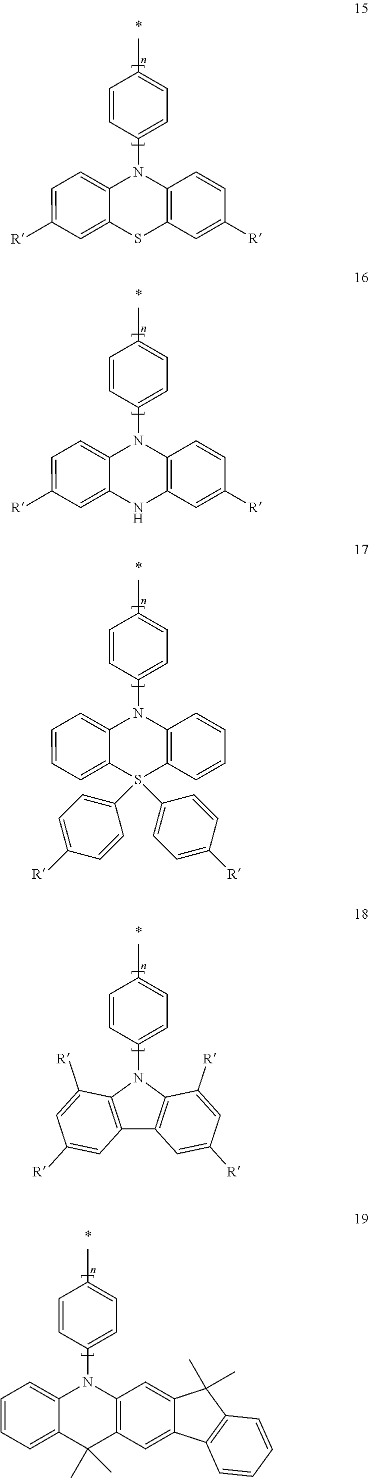

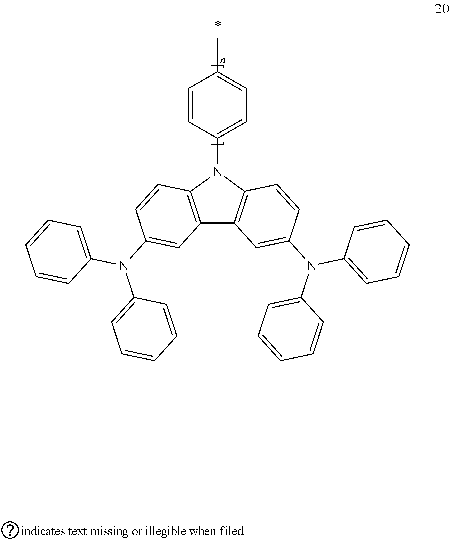

where, R′ is a hydrogen atom, a tert-butyl group, a methoxy group, a cyano group, a fluorine atom or an alkyl chain, n is a natural number from 0 to 10, and * is a substitution position; and the R₂ is one of the following substitutional groups from a to o:

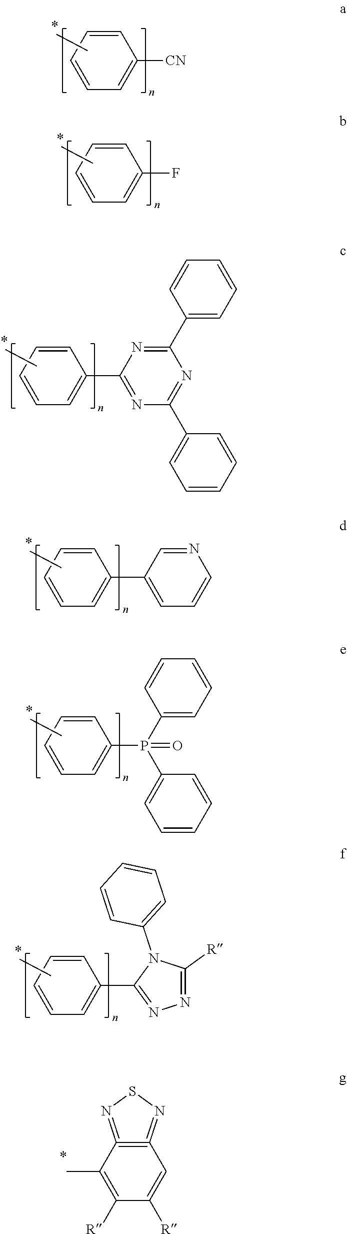

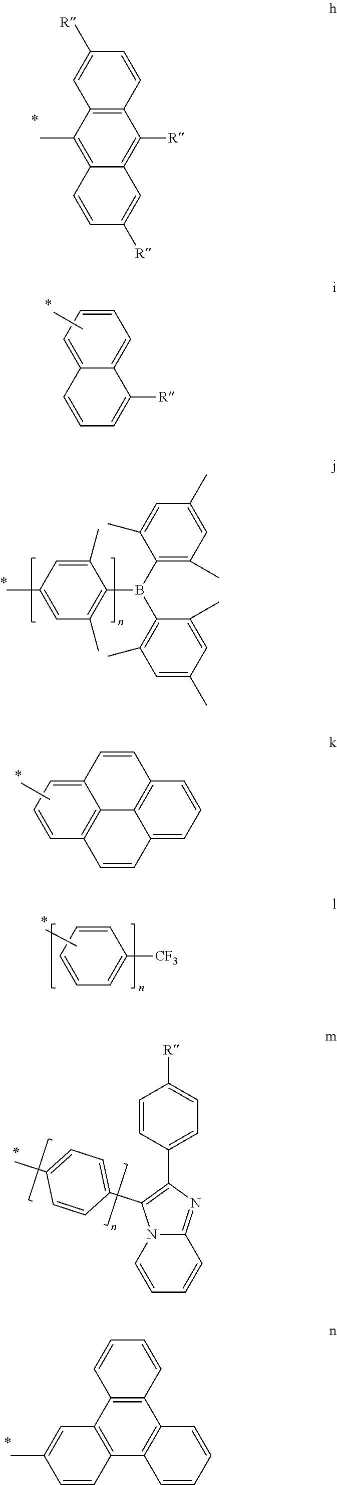

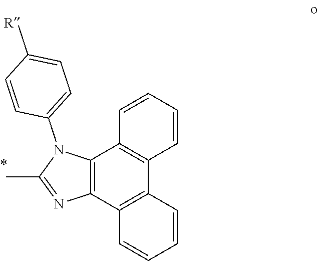

where, R″ is a hydrogen atom, a tert-butyl group, a methoxy group, a cyano group, a fluorine atom or an alkyl chain, n is a natural number from 0 to 10, and * is a substitution position.

Further, the alkyl chain refers to a straight chain, a branched chain or a cyclicalkyl chain that has 1 to 20 carbon atoms, or an alkyl chain of which one or more carbon atoms are substituted with an oxygen atom, an alkenyl group, an alkynyl group, an aryl group, a carbonyl group, a hydroxyl group, an amino group, a carboxyl group, a cyano group, a nitro group or an ester group, or an alkyl chain of which one or more hydrogen atoms are substituted with a fluorine atom, a chlorine atom, a bromine atom or an iodine atom.

A preparation method of the above tetraphenylbenzene-containing organic light-emitting material includes the following steps:
using 1,4-dibromo-2,5-terphenyl and R₂-substituted phenylboronic acid as raw materials, obtaining an R₂-containing aromatic ring compound through a Suzuki reaction; and then reacting the R₂-containing aromatic ringcompound with R₁-substituted phenylboronic acid or boronic acid ester in the presence of a tetrakis(triphenylphosphine)palladium catalyst to obtain the tetraphenylbenzene-containing organic light-emitting material, the above preparation method having a reaction formula as follows:

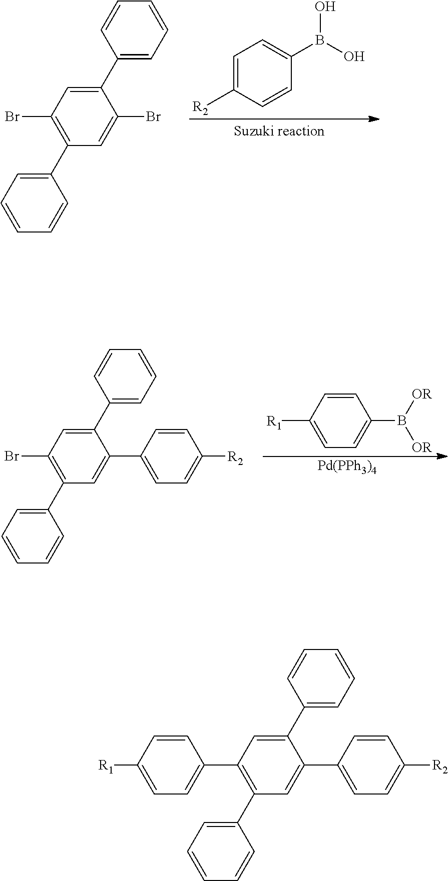

The above tetraphenylbenzene-containing organic light-emitting material is applied to organic light-emitting devices.

The present disclosure regulates the carrier transport performance of a tetraphenylbenzene derivative well by binding different electron-donating groups and electron-withdrawing groups to tetraphenylbenzene. The obtained structure is contorted and hardly produces strong 7L-7L interaction in the aggregation state; in addition, this contorted molecular structure is beneficial to improving the triplet energy level of the material, so that the obtained material can be used as a host material for emitting light of different colors. Characterization data of organic light-emitting devices can show that the tetraphenylbenzene-containing organic light-emitting material of the present disclosure can be not only used as a light-emitting layer, but also used as a phosphorescent host material so as to prepare light-emitting devices with good optoelectronic performance, simple structure, and low cost, and therefore, the tetraphenylbenzene-containing organic light-emitting material has broad application prospects in the field of organic electroluminescence, and is expected to be widely used in the fields of flat panel display and solid-state lighting.

Compared with the prior art, the present disclosure has the following advantages and beneficial effect:
(1) the tetraphenylbenzene-containing organic light-emitting material of the present disclosure has the features of efficient solid-stateluminescence and bipolarity, and can be used to prepare non-doped blue organic light-emitting devices with high efficiency and low roll-off,
(2) the synthesis method of the tetraphenylbenzene-containing organic light-emitting material of the present disclosure is simple, the raw materials are readily available, the yield is high, and the obtained material has a stable structure and is easy to store; and
(3) the tetraphenylbenzene-containing organic light-emitting material has excellent illuminance performance and can be widely applied to the fields such as organic electroluminescence.

### BRIEF DESCRIPTION OF THE DRAWINGS

FIG. 1 is a J-V-L curve chart of a non-doped blue OLED prepared by a tetraphenylbenzene-containing organic light-emitting material according to Example 1;

FIG. 2 is an efficiency-brightness curve chart of the non-doped blue OLED prepared by the tetraphenylbenzene-containing organic light-emitting material according to Example 1;

FIG. 3 is a J-V-L curve chart of a mixed white OLED prepared by the tetraphenylbenzene-containing organic light-emitting material according to Example 1;

FIG. 4 is an efficiency-brightness curve chart of the mixed white OLED prepared by the tetraphenylbenzene-containing organic light-emitting material according to Example 1;

FIG. 5 is a J-V-L curve chart of a non-doped blue OLED prepared by a tetraphenylbenzene-containing organic light-emitting material according to Example 2; and

FIG. 6 is an efficiency-brightness curve chart of the non-doped blue OLED prepared by the tetraphenylbenzene-containing organic light-emitting material according to Example 2.

### DETAILED DESCRIPTION OF THE EMBODIMENTS

The present disclosure will be further described below in detail with reference to the embodiments and drawings, but the implementation modes of the present disclosure are not limited thereto.

### Example 1

A tetraphenylbenzene-containing organic light-emitting material (TPA-TPB-CN) was prepared in the present example:

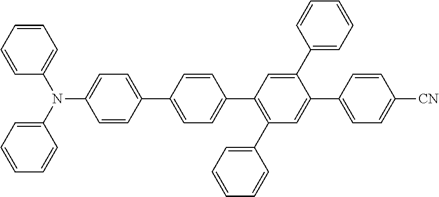 [SMILES: N#Cc1ccc(-c2cc(-c3ccccc3)c(-c3ccc(-c4ccc(N(c5ccccc5)c5ccccc5)cc4)cc3)cc2-c2ccccc2)cc1]

A synthesis route is as follows:

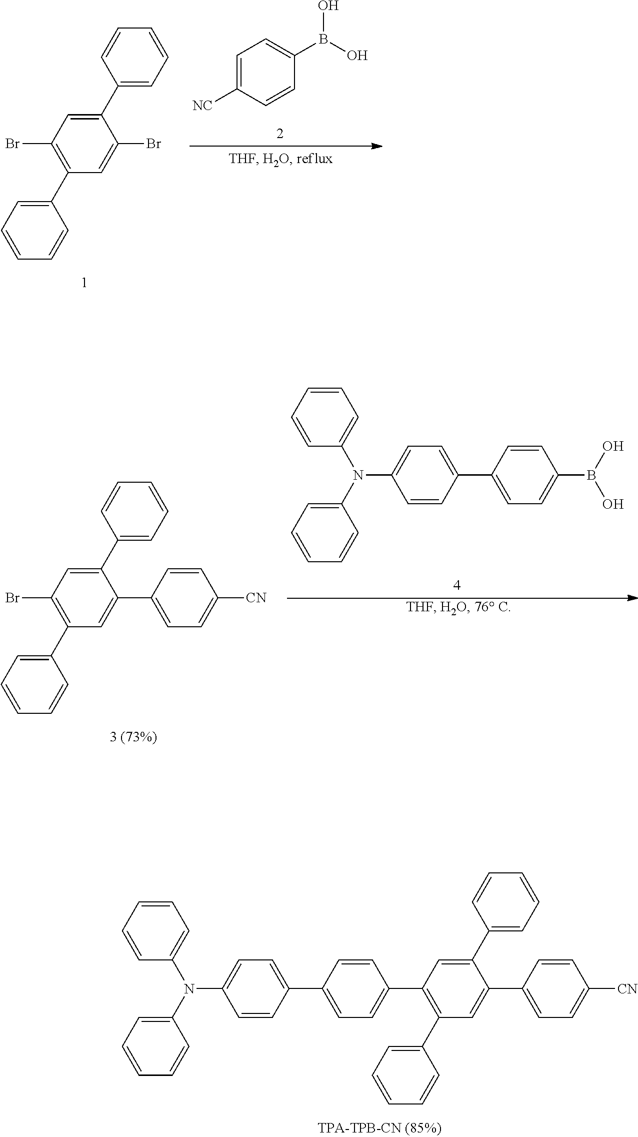 [SMILES: Brc1cc(-c2ccccc2)c(Br)cc1-c1ccccc1 ; N#Cc1ccc(B(O)O)cc1 ; N#Cc1ccc(-c2cc(-c3ccccc3)c(Br)cc2-c2ccccc2)cc1 ; OB(O)c1ccc(-c2ccc(N(c3ccccc3)c3ccccc3)cc2)cc1 ; "THF, H2O, 76° C." ; N#Cc1ccc(-c2cc(-c3ccccc3)c(-c3ccc(-c4ccc(N(c5ccccc5)c5ccccc5)cc4)cc3)cc2-c2ccccc2)cc1]

(1) bromo-p-terphenyl (compound 1) (6 g, 15.5 mmol), 4-cyanophenylboronic acid (compound 2) (2.50 g, 17.0 mmol), anhydrous potassium carbonate (6.4 g, 46.5 mmol), and Pd(PPh₃)₄ (895 mg, 0.8 mmol) were added into a 250 mL reaction flask, 105 mL of THF and 15 mL of water were added under the protection of nitrogen, and the reaction mixture was refluxed overnight, cooled, extracted with dichloromethane, concentrated, ground into powder, and subjected to chromatographic separation to prepare a white solid (intermediate 3) with the yield of 73%; and
(2) the intermediate 3 (2.136 g, 1.5 mmol), 4-(diphenylamino)phenylboronic acid (compound 4) (1.6 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid TPA-TPB-CN with the yield of 85%; and data of identification of the product areas follows:

¹H NMR (MHz),δ (TMS, ppm): 7.59 (s, 1H), 7.55 (t, 1H), 7.53 (t, 1H), 7.51 (d, 3H), 7.48 (d, 2H), 7.37 (t, 1H), 7.35 (t, 1H), 7.26 (m, 14H), 7.22 (m, 2H), 7.10 (m, 6H), 7.03 (m, 2H).

### Example 2

A tetraphenylbenzene-containing organic light-emitting material (Cz-TPB-CN) was prepared in the present example:

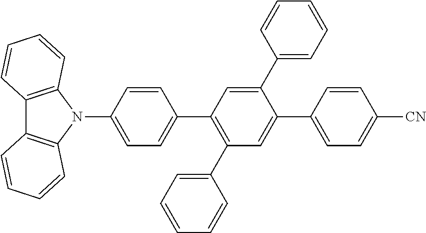 [SMILES: N#Cc1ccc(-c2cc(-c3ccccc3)c(-c3ccc(-n4c5ccccc5c5ccccc54)cc3)cc2-c2ccccc2)cc1]

A synthesis route is as follows:

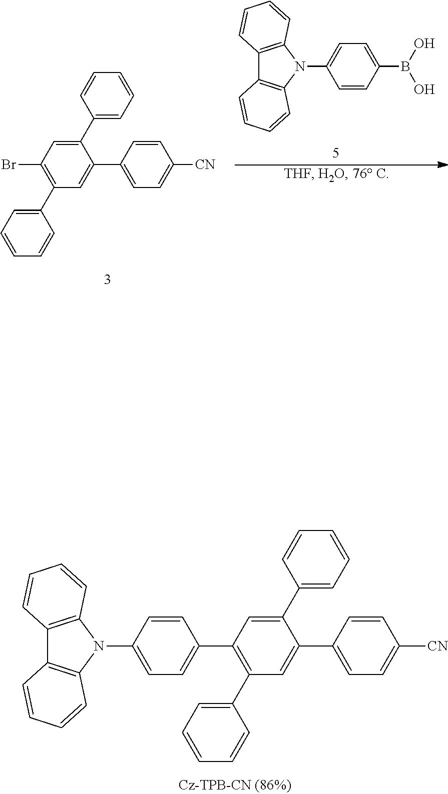 [SMILES: N#Cc1ccc(-c2cc(-c3ccccc3)c(Br)cc2-c2ccccc2)cc1 ; OB(O)c1ccc(-n2c3ccccc3c3ccccc32)cc1 ; "THF, H2O, 76° C." ; N#Cc1ccc(-c2cc(-c3ccccc3)c(-c3ccc(-n4c5ccccc5c5ccccc54)cc3)cc2-c2ccccc2)cc1]

the intermediate 3 (2.136 g, 1.5 mmol), 4-boronic acid carbazole (compound 5) (1.119 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid Cz-TPB-CN with the yield of 86%; and data of identification of the product areas follows:

¹H NMR (500 MHz, CD₂Cl₂), δ (TMS, ppm): 8.16 (m, 1H), 8.14 (m, 1H), 7.69 (s, 1H), 7.58 (d, 2H), 7.56 (m, 1H), 7.47 (d, 4H), 7.45-7.38 (m, 6H), 7.35-7.25 (m, 12H).

### Example 3

Atetraphenylbenzene-containing organic light-emitting material (3PhCz-TPB-CN) was prepared in the present example:

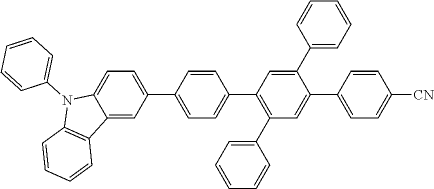 [SMILES: N#Cc1ccc(-c2cc(-c3ccccc3)c(-c3ccc(-c4ccc5c(c4)c4ccccc4n5-c4ccccc4)cc3)cc2-c2ccccc2)cc1]

A synthesis route is as follows:

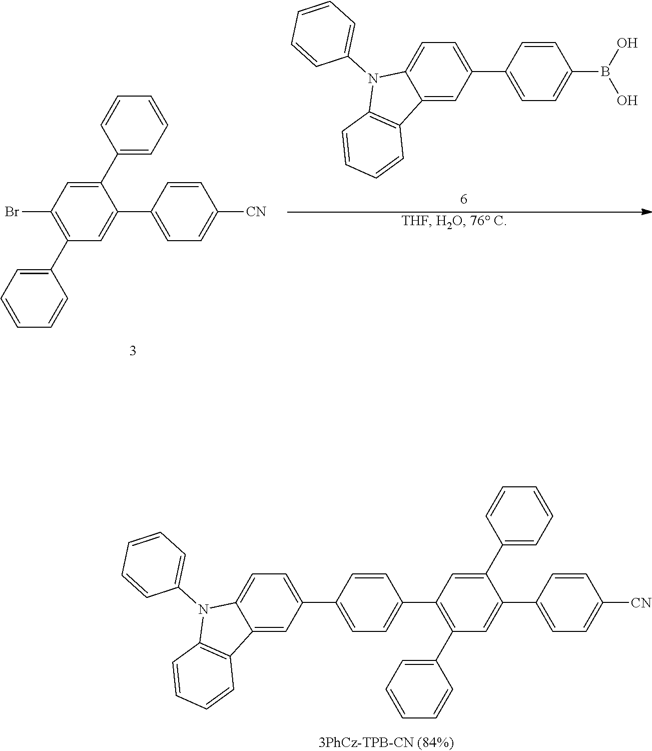 [SMILES: N#Cc1ccc(-c2cc(-c3ccccc3)c(Br)cc2-c2ccccc2)cc1 ; OB(O)c1ccc(-c2ccc3c(c2)c2ccccc2n3-c2ccccc2)cc1 ; "THF, H2O, 76° C." ; N#Cc1ccc(-c2cc(-c3ccccc3)c(-c3ccc(-c4ccc5c(c4)c4ccccc4n5-c4ccccc4)cc3)cc2-c2ccccc2)cc1]

the intermediate 3 (2.136 g, 1.5 mmol), the compound 6 (1.415 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid 3PhCz-TPB-CN with the yield of 84%; and data of identification of the product areas follows:

¹H NMR (500 MHz, CDCl₃), δ (TMS, ppm): 8.35 (d, 1H), 8.18 (m, 1H), 7.59-7.66 (m, 8H), 7.50 (d, 3H), 7.47 (m, 4H), 7.27-7.45 (d, 13H), 7.29 (m, 2H).

### Example 4

A tetraphenylbenzene-containing organic light-emitting material (AD-TPB-CN) was prepared in the present example:

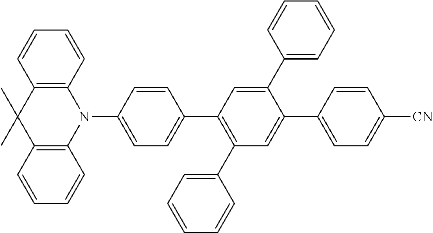 [SMILES: CC1(C)c2ccccc2N(c2ccc(-c3cc(-c4ccccc4)c(-c4ccc(C#N)cc4)cc3-c3ccccc3)cc2)c2ccccc21]

A synthesis route is as follows:

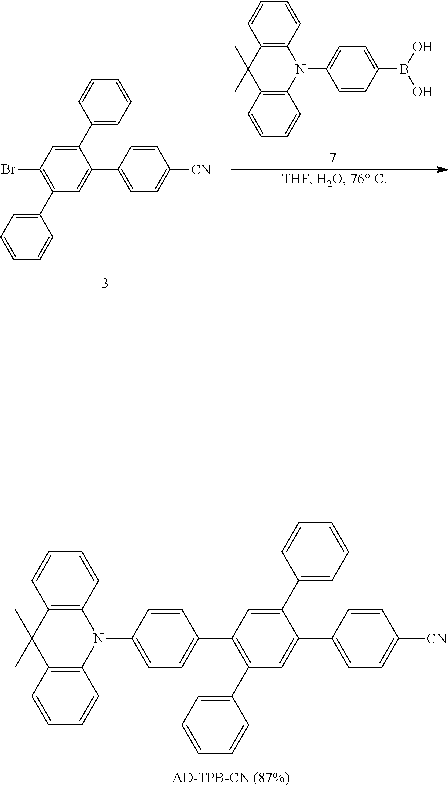 [SMILES: N#Cc1ccc(-c2cc(-c3ccccc3)c(Br)cc2-c2ccccc2)cc1 ; CC1(C)c2ccccc2N(c2ccc(B(O)O)cc2)c2ccccc21 ; "THF, H2O, 76° C." ; CC1(C)c2ccccc2N(c2ccc(-c3cc(-c4ccccc4)c(-c4ccc(C#N)cc4)cc3-c3ccccc3)cc2)c2ccccc21]

the intermediate 3 (2.136 g, 1.5 mmol), the compound 7 (1.283 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid AD-TPB-CN with the yield of 87%; and data of identification of the product are as follows:

¹H NMR (400 MHz),δ (TMS, ppm): 7.69 (s, 1H), 7.58 (d, 2H), 7.56 (t, 1H), 7.47 (t, 4H), 7.40 (t, 1H), 7.38 (t, 1H), 7.33-7.25 (m, 10H), 7.22 (d, 1H), 7.20 (s, 1H), 6.97 (m, 4H), 6.26 (m, 2H), 1.66 (m, 6H).

### Example 5

Atetraphenylbenzene-containing organic light-emitting material (CzPh-TPB-CN) was prepared in the present example:

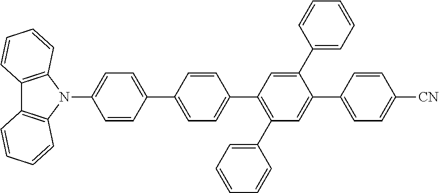 [SMILES: N#Cc1ccc(-c2cc(-c3ccccc3)c(-c3ccc(-c4ccc(-n5c6ccccc6c6ccccc65)cc4)cc3)cc2-c2ccccc2)cc1]

A synthesis route is as follows:

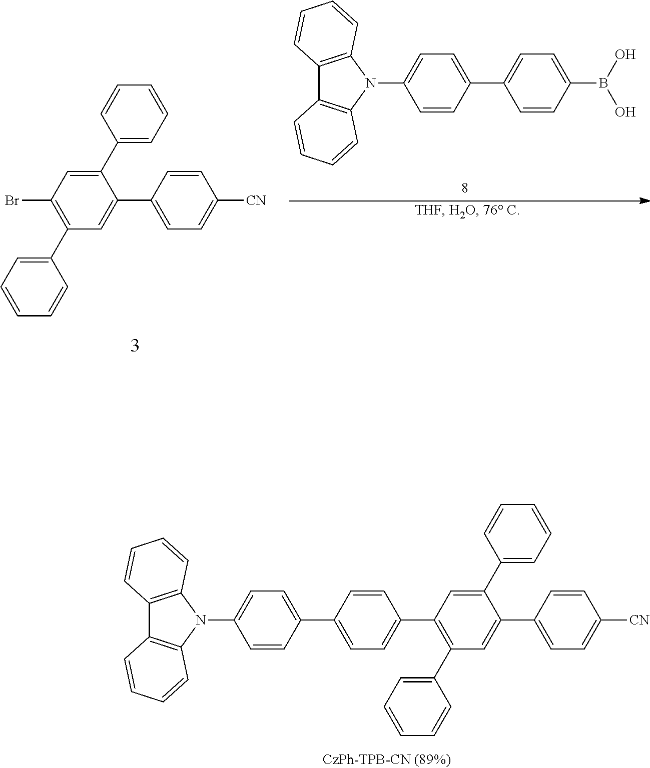 [SMILES: N#Cc1ccc(-c2cc(-c3ccccc3)c(Br)cc2-c2ccccc2)cc1 ; OB(O)c1ccc(-c2ccc(-n3c4ccccc4c4ccccc43)cc2)cc1 ; "THF, H2O, 76° C." ; N#Cc1ccc(-c2cc(-c3ccccc3)c(-c3ccc(-c4ccc(-n5c6ccccc6c6ccccc65)cc4)cc3)cc2-c2ccccc2)cc1]

the intermediate 3 (2.136 g, 1.5 mmol), the compound 8 (1.416 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid CzPh-TPB-CN with the yield of 89%.

### Example 6

A tetraphenylbenzene-containing organic light-emitting material (DPA-TPB-CN) was prepared in the present example:

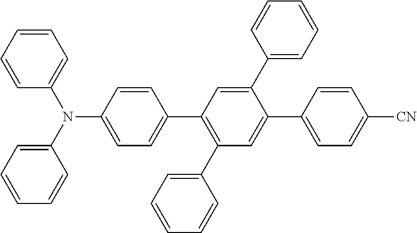 [SMILES: N#Cc1ccc(-c2cc(-c3ccccc3)c(-c3ccc(N(c4ccccc4)c4ccccc4)cc3)cc2-c2ccccc2)cc1]

A synthesis route is as follows:

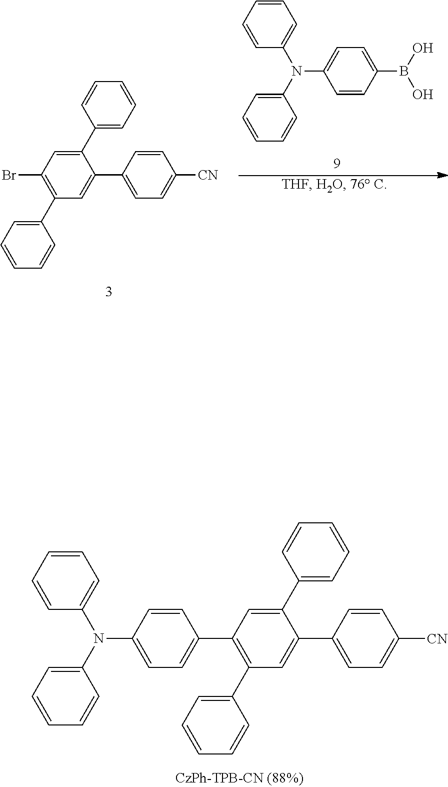 [SMILES: N#Cc1ccc(-c2cc(-c3ccccc3)c(Br)cc2-c2ccccc2)cc1 ; OB(O)c1ccc(N(c2ccccc2)c2ccccc2)cc1 ; "THF, H2O, 76° C." ; N#Cc1ccc(-c2cc(-c3ccccc3)c(-c3ccc(N(c4ccccc4)c4ccccc4)cc3)cc2-c2ccccc2)cc1]

the intermediate 3 (2.136 g, 1.5 mmol), the compound 9 (1.127 g, 3.9 mmol), anhydrous potassium carbonate (1.616 g, 11.7 mmol), and Pd(PPh₃)₄ (225 mg, 0.195 mmol) were added into a 250 mL reaction flask, 10 mL of THF and 5 mL of water were added under the protection of nitrogen, and the reaction mixture was refluxed overnight, cooled, extracted with dichloromethane, concentrated, ground into powder, and subjected to chromatographic separation to prepare a white solid DPA-TPB-CN with the yield of 88%.

### Example 7

A tetraphenylbenzene-containing organic light-emitting material (Cz-TPB-3Q) was prepared in the present example:

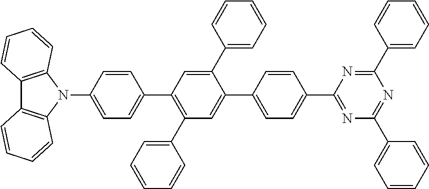 [SMILES: c1ccc(-c2nc(-c3ccccc3)nc(-c3ccc(-c4cc(-c5ccccc5)c(-c5ccc(-n6c7ccccc7c7ccccc76)cc5)cc4-c4ccccc4)cc3)n2)cc1]

A synthesis route is as follows:

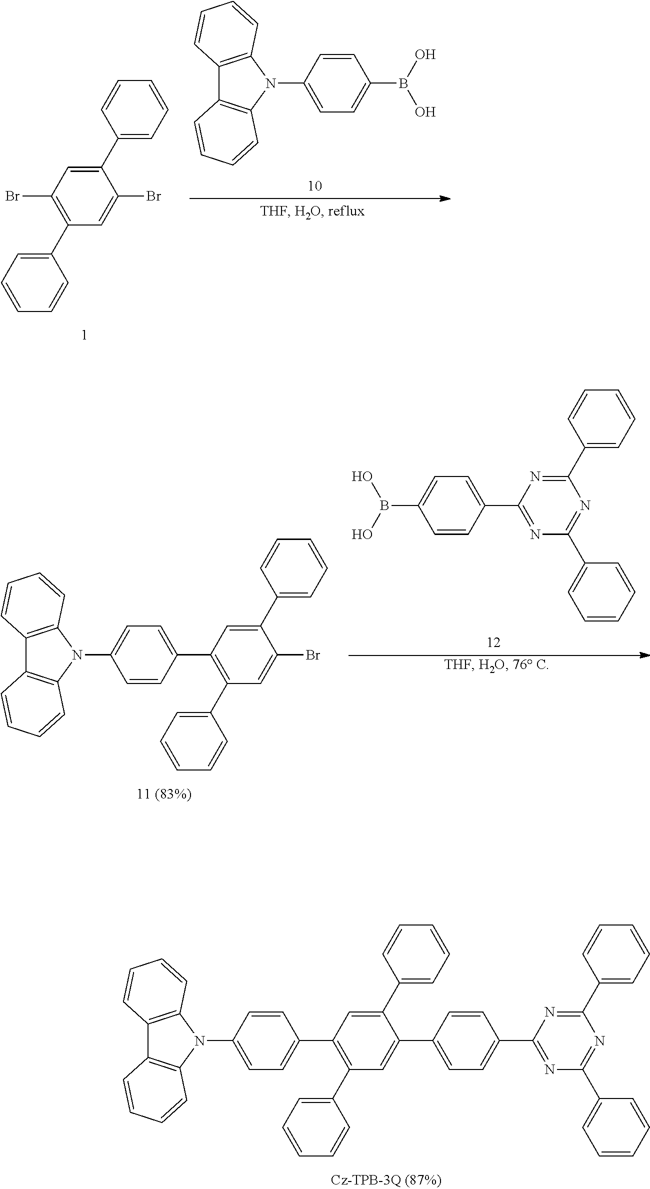

(1) bromo-p-terphenyl (compound 1) (5.43 g, 14.0 mmol), carbazoleylphenylboronic acid (compound 10) (4.82 g, 16.8 mmol), anhydrous potassium carbonate (5.8 g, 42.0 mmol), and Pd(PPh₃)₄ (809 mg, 0.7 mmol) were added into a 250 mL reaction flask, 90 mL of THF and 21 mL of water were added under the protection of nitrogen, and the reaction mixture was refluxed overnight, cooled, extracted with dichloromethane, concentrated, ground into powder, and subjected to chromatographic separation to prepare a white solid (intermediate 11) with the yield of 91%; and
(2) the intermediate 11 (1.10 g, 2 mmol), 4-boronicacid-1,3,5-triphenyltriazine (compound 12) (847 mg, 2.4 mmol), anhydrous potassium carbonate (828 mg, 6.0 mmol), and Pd(PPh₃)₄ (116 mg, 0.10 mmol) were added into a 125 mL reaction flask, 14 mL of THF and 3 mL of water were added under the protection of nitrogen, and the reaction mixture was refluxed overnight, cooled, extracted with dichloromethane, concentrated, ground into powder, and subjected to chromatographic separation to prepare a white solid TPA-TPB-3Q with the yield of 84%.

### Example 8

The performance of a blue OLED prepared by a tetraphenylbenzene-containing organic light-emitting material (TPA-TPB-CN) was tested in the present example.

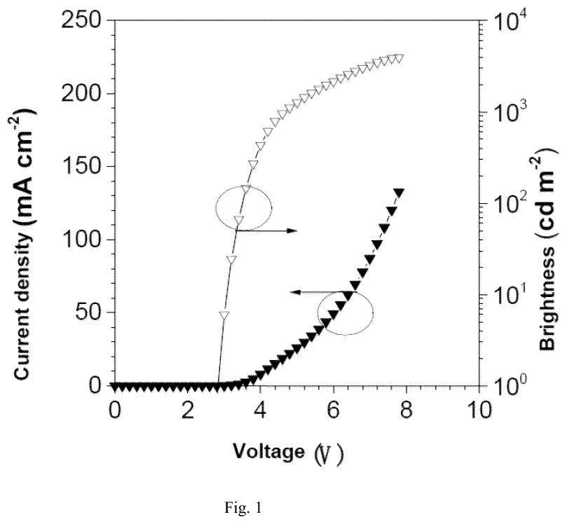

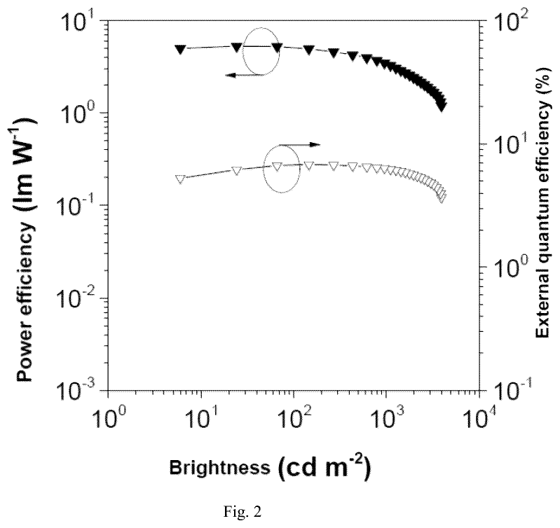

The tetraphenylbenzene-containing organic light-emitting material TPA-TPB-CN (the solid-state fluorescence quantum yield=93.2%) of Example 1 was used as a light-emitting material to prepare a non-doped blue device, the performance of the device was tested and characterized, and results are shown in FIG. 1 and FIG. 2.

Device structure: ITO/HAT-CN (5 nm)/TAPC (50 nm)/TCTA (5 nm)/TPA-TPB-CN (20 nm)/TmPyPB (40 nm)/LiF (1 nm)/Al.

FIG. 1 is a J-V-L curve chart of the non-doped blue OLED prepared by the tetraphenylbenzene-containing organic light-emitting material of Example 1. It can be seen from the figure that the TPA-TPB-CN-based non-doped device has high maximum brightness and low starting voltage, which are 3,945 cd/m² and 2.8 V, respectively.

FIG. 2 is an efficiency-brightness curve chart of the non-doped blue OLED prepared by the tetraphenylbenzene-containing organic light-emitting material of Example 1. It can be seen from the figure that the TPA-TPB-CN-based non-doped device has high efficiency, low roll-off, and the maximum external quantum efficiency of 6.8%; when the brightness is 1,000 cd/m², the external quantum efficiency still maintains at 6.33%; and the emission wavelength is 446 nm.

### Example 9

The performance of a mixed white OLED prepared by a tetraphenylbenze-containing organic light-emitting material (TPA-TPB-CN) was tested in the present example.

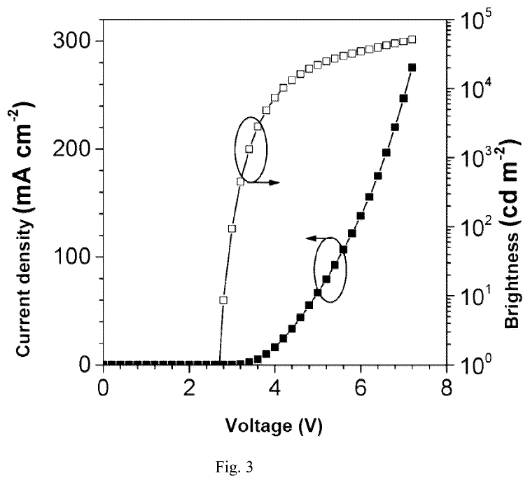

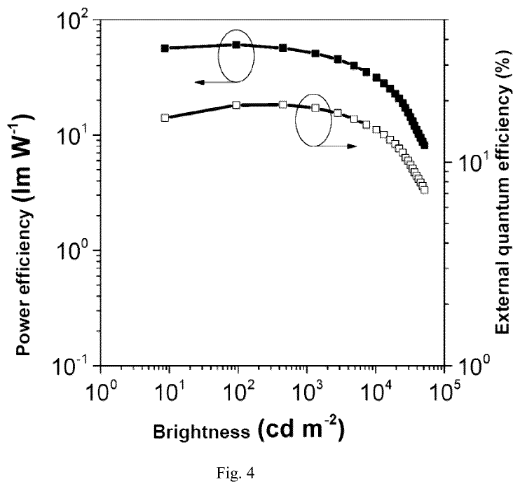

The tetraphenylbenzene-containing organic light-emitting material TPA-TPB-CN of Example 1 was used as a blue light-emitting layer and a yellow phosphorescentbody to prepare a double-color white device, the performance of the device was tested and characterized, and results are shown in FIG. 3 and FIG. 4.

Device structure: ITO/HAT-CN (5 nm)/TAPC (50 nm)/TCTA (5 nm)/TPA-TPB-CN (8 nm)/TPA-TPB-CN: 3% PO-01 (12 nm)/TmPyPB (40 nm)/LiF (1 nm)/Al.

FIG. 3 is a J-V-L curve chart of the mixed white OLED prepared by the tetraphenylbenzene-containing organic light-emitting material (TPA-TPB-CN) of Example 1. It can be seen from the figure that the obtained device has high maximum brightness and low starting voltage, which are 51,393 cd/m² and 2.8 V, respectively.

FIG. 4 is an efficiency-brightness curve chart of the double-color white OLED prepared by the tetraphenylbenzene-containing organic light-emitting material (TPA-TPB-CN) of Example 1. It can be seen from the figure that the obtained double-color white device has high efficiency, low roll-off, the maximum power efficiency of 60.7 lm/W, and the maximum external quantum efficiency of 19.1%; and when the brightness is 1,000 cd/m², the external quantum efficiency is 18.7%.

### Example 10

The performance of a non-doped dark blue OLED prepared by a tetraphenylbenzene-containing organic light-emitting material (Cz-TPB-CN) was tested in the present example.

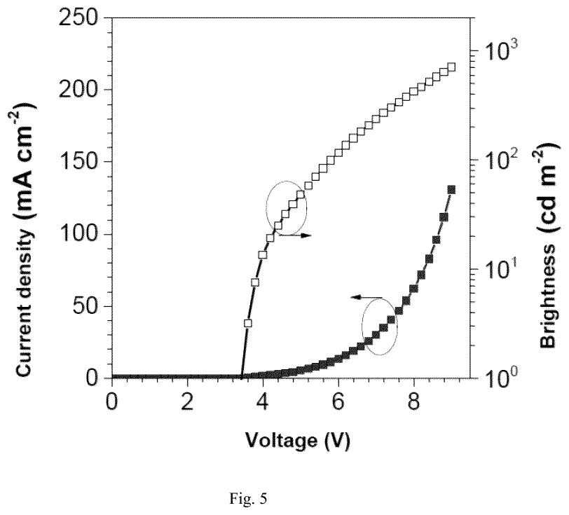

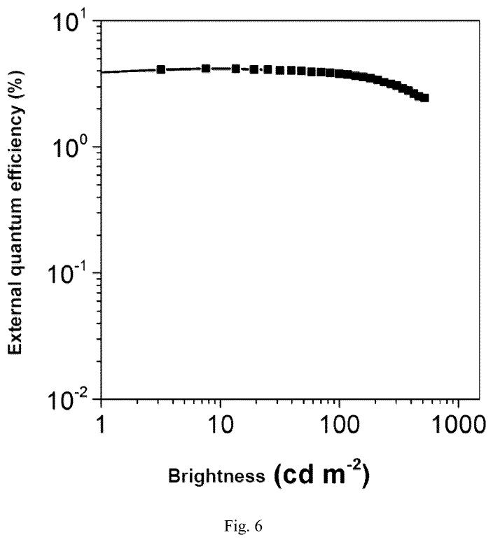

The tetraphenylbenzene-containing organic light-emitting material Cz-TPB-CN (the solid-state fluorescence quantum yield=99.9%) of Example 2 was used as a light-emitting material to prepare a non-doped blue device, the performance of the device was tested and characterized, and results are shown in FIG. 5 and FIG. 6.

Device structure: ITO/HAT-CN (5 nm)/TAPC (50 nm)/mCP (5 nm)/Cz-TPB-CN (20 nm)/TmPyPB (40 nm)/LiF (1 nm)/Al.

FIG. 5 is a J-V-L curve chart of the non-doped blue OLED prepared by the tetraphenylbenzene-containing organic light-emitting material of Example 2. It can be seen from the figure that the obtained device has the maximum brightness of 709 cd/m² and a starting voltage of 3.4 V.

FIG. 6 is an efficiency-brightness curve chart of the non-doped blue OLED prepared by the tetraphenylbenzene-containing organic light-emitting material of Example 2. It can be seen from the figure that the obtained device has high efficiency, low roll-off, and the maximum external quantum efficiency of 4.17%; and when the brightness is 100 cd/m², the external quantum efficiency is 3.80%.

The above data show that the present disclosure obtains dark blue light-emitting molecules with AIE performance by binding different electron-donating groups to tetraphenylbenzene, and non-doped dark blue OLEDs prepared by using these materials as light-emitting layers have high efficiency and low roll-off, the non-doped OLEDs which have simple structures and are prepared based on these materials have lower starting voltage, higher efficiency, lower roll-off; and meanwhile, these materials can also be used as bodies to prepare double-color white devices, and the obtained devices have high efficiency and low roll-off. In conclusion, these materials have very broad application prospects in the field of organic electroluminescence.

The above embodiments are preferred implementation modes of the present disclosure, but the implementation modes of the present disclosure are not limited thereto, any change, modification, substitution, combination or simplification made without departing from the spirit essence and principle of the present disclosure shall be regarded as equivalent replacement and shall fall within the scope of protection of the present disclosure.

1-5. (canceled)

6: A tetraphenylbenzene-containing organic light-emitting material, having a structural formula as Formula I: 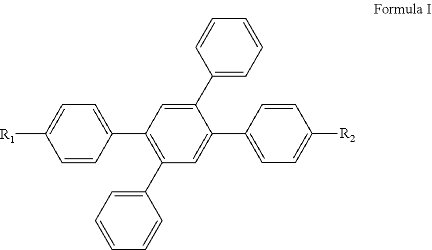
the R₁ is 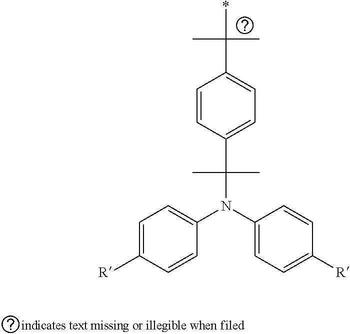
where, R′ is a hydrogen atom, a methoxy group, a cyano group, a fluorine atom or an alkyl chain, n is a natural number from 1 to 10, and * is a substitution position; and
the R₂ is 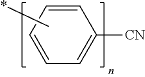
where, R″ is a hydrogen atom, a methoxy group, a cyano group, a fluorine atom or an alkyl chain, n is a natural number from 1 to 10, and * is a substitution position.

7: The tetraphenylbenzene-containing organic light-emitting material according to claim 6, characterized in that the alkyl chain refers to a straight chain, a branched chain or a cyclic alkyl chain that has 1 to 20 carbon atoms, or an alkyl chain of which one or more carbon atoms are substituted with an oxygen atom, an alkenyl group, an alkynyl group, an aryl group, a carbonyl group, a hydroxyl group, an amino group, a carboxyl group, a cyano group, a nitro group or an ester group, or an alkyl chain of which one or more hydrogen atoms are substituted with a fluorine atom, a chlorine atom, a bromine atom or an iodine atom.

8: A preparation method of the tetraphenylbenzene-containing organic light-emitting material according to claim 6, comprising the following steps:
using 1,4-dibromo-2,5-terphenyl and R₂-substituted phenylboronic acid as raw materials, obtaining an R₂-containing aromatic ring compound through a Suzuki reaction; and then reacting the R₂-containing aromatic ring compound with R₁-substituted phenylboronic acid or boronic acid ester in the presence of a tetrakis(triphenylphosphine)palladium catalyst to obtain the tetraphenylbenzene-containing organic light-emitting material, and the above preparation method having a reaction formula as follows:

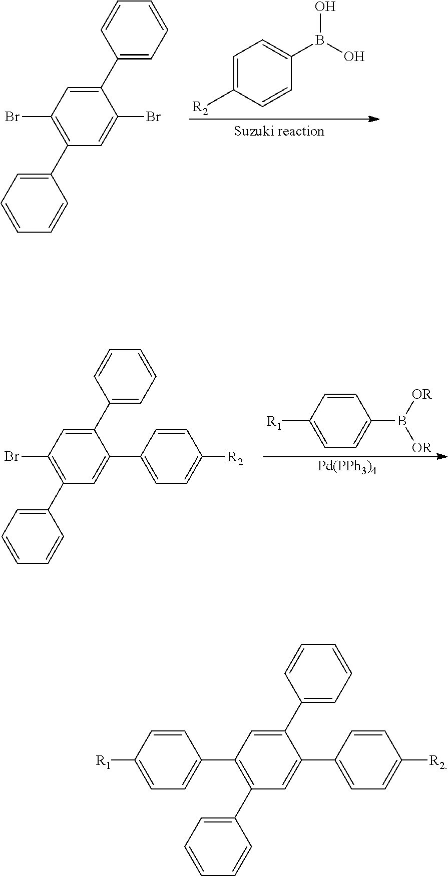

9: A preparation method of the tetraphenylbenzene-containing organic light-emitting material according to claim 7, comprising the following steps:
using 1,4-dibromo-2,5-terphenyl and R₂-substituted phenylboronic acid as raw materials, obtaining an R₂-containing aromatic ring compound through a Suzuki reaction; and then reacting the R₂-containing aromatic ring compound with R₁-substituted phenylboronic acid or boronic acid ester in the presence of a tetrakis(triphenylphosphine)palladium catalyst to obtain the tetraphenylbenzene-containing organic light-emitting material, and the above preparation method having a reaction formula as follows:

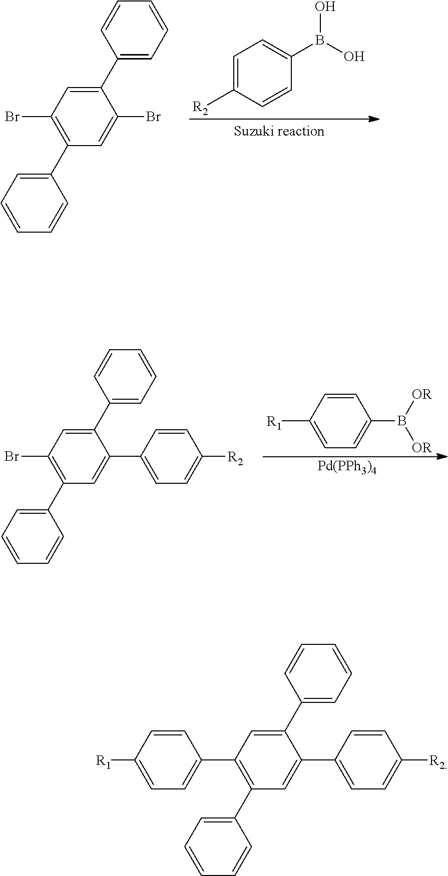

10: An application of the tetraphenylbenzene-containing organic light-emitting material according to claim 6 in organic light-emitting devices.

11: An application of the tetraphenylbenzene-containing organic light-emitting material according to claim 7 in organic light-emitting devices.
